(19) United States
(12) Patent Application Publication
(10) Pub. No.: US 2014/0248228 A1
(43) Pub. Date: Sep. 4, 2014

(54) HAIR TREATMENT COMPOSITION WITH CATIONIC CARE SUBSTANCE AND UV FILTER

(71) Applicant: Henkel AG & Co. KGaA, Duesseldorf (DE)

(72) Inventor: Marcus Krueger, Ellerhoop (DE)

(73) Assignee: Henkel AG & Co. KGaA, Duesseldorf (DE)

(21) Appl. No.: 14/278,294

(22) Filed: May 15, 2014

Related U.S. Application Data
(63) Continuation of application No. PCT/EP2012/071123, filed on Oct. 25, 2012.

(30) Foreign Application Priority Data
Nov. 18, 2011 (DE) 10 2011 086 661.2

(51) Int. Cl. A61K 8/45; A61K 8/35; A61K 8/58; A61K 8/44; A61K 8/37; A61K 8/29; A61Q 5/00; A61K 8/49

(52) U.S. Cl. CPC A61K 8/45; A61Q 5/00; A61K 8/35; A61K 8/4946; A61K 8/445; A61K 8/37; A61K 8/29; A61K 8/585
USPC 424/70.9

(57) ABSTRACT

Hair treatment agents combining improved care effects, in particular improved combability, softness and shine of the hair, with a viscosity stability over wide temperature ranges include—relative to their weight—0.1 to 20 wt. % of at least one UV filter and 0.1 to 30 wt. % of at least one compound of the general formula (I), in which n and m independently of one another denote integers between 5 and 40, with the proviso that n+m≧38; a and b independently of one another denote integers between 1 and 10; R and R′ are selected independently of one another from —H and —CH₃; X⁻ is a physiologically acceptable anion.

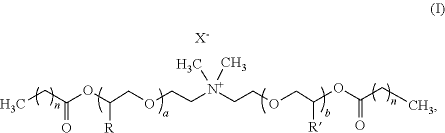

### FIELD OF THE INVENTION

The present invention generally relates to hair treatment agents including cationic care substance(s) and (a) certain UV filter(s) and to the methods for cleansing and/or caring for hair using these agents.

### BACKGROUND OF THE INVENTION

The cosmetic treatment of skin and hair is an important element of human body care. Thus human hair is now treated in many different ways with hair cosmetics preparations. They include cleansing the hair with shampoos, care and regeneration with rinses and masks, and bleaching, coloring and shaping the hair with coloring agents, tints, permanent wave agents and styling preparations. Agents for changing or shading the color of the head hair have a prominent role to play here.

Not least because of the heavy loading on the hair, resulting for example from coloring or permanent waving and from cleansing the hair with shampoos as well as from environmental pollution, the importance of care products having as long-lasting an effect as possible is growing. Such care agents influence the natural structure and properties of the hair. Thus, following such treatments, the wet and dry combability of the hair or the hold and fullness of the hair can be optimized, for example, or the hair can be protected from the increased occurrence of split ends. Moreover, combined preparations have recently been developed to reduce the time spent on the usual multi-stage methods, in particular when directly applied by consumers. In addition to the usual components, for cleansing the hair for example, these preparations additionally include active ingredients that previously were reserved for hair aftertreatment agents. The consumer is thus spared an application step; at the same time the packaging cost is reduced, as one less product is used.

As a general rule the active ingredients that are available both for separate aftertreatment agents and for combined preparations preferably act on the surface of the hair. Thus, active ingredients which impart shine, hold, fullness or improved wet or dry combability to the hair or which prevent split ends are known. However, just as significant as the external appearance of the hair is the internal structural cohesion of the hair fibers, which in the case in particular of oxidative and reductive processes such as coloring and permanent waving can be strongly influenced.

However, the known active ingredients cannot cover all requirements to an adequate extent. There is therefore still a demand for active ingredients or for combinations of active ingredients for cosmetic agents having good care properties and good biodegradability. In formulations including dyes and/or electrolytes in particular there is a need for additional caring active ingredients that are able to be incorporated without difficulty into known formulations.

Owing to the affinity to keratin fibers, cationic care substances have a particular significance. Numerous substances from the most diverse groups are known and are widely used in cosmetic agents.

Thus EP 951 898 B1 discloses hair-conditioning agents including quaternary ammonium compounds from the group of esterquats and at least one silicone compound.

Diesterquats, i.e. quaternary ammonium compounds having two acyl residues in the molecule, are disclosed for example in EP 918 743.

WO 2004/093834 A1 discloses special diesterquats which are based on diols rather than ethanolamines and have long-chain acyl residues, which can optionally be bound to the diol groupings by means of PEG or PPG groupings.

It is therefore desirable to provide hair treatment agents combining improved care effects, in particular improved combability, softness and shine of the hair, with a viscosity stability over wide temperature ranges.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A hair treatment agent including—relative to its total weight—a) at least one UV filter in a total amount from 0.01 to 20 wt. %; b) at least one compound of the general formula (I) in a total amount from 0.1 to 30 wt. %

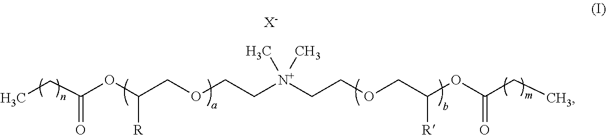

in which n and m independently of one another denote integers between 5 and 40, with the proviso that n+m≧38; a and b independently of one another denote integers between 1 and 10; R and R′ are selected independently of one another from —H and —CH₃; X⁻ is a physiologically acceptable anion.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that the combination of cationic care substances and certain UV filter substances can achieve the specified advantages.

The present invention therefore provides in a first embodiment a hair treatment agent including—relative to its total weight—
a) at least one IN filter in a total amount from 0.1 to 20 wt. %
b) at least one compound of the general formula (I) in a total amount from 0.1 to 30 wt. %

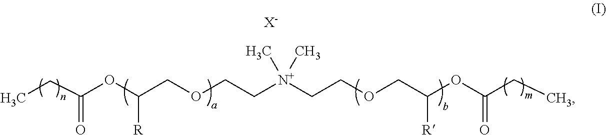

in which
n and m independently of one another denote integers between 5 and 40, with the proviso that n+m≧38;
a and b independently of one another denote integers between 1 and 10;
R and R′ are selected independently of one another from —H and —CH₃;
X⁻ is a physiologically acceptable anion.

Hair treatment agents within the meaning of the present invention are for example hair coloring agents, bleaching agents, hair shampoos, hair conditioners, conditioning shampoos, hair sprays, hair rinses, hair masks, hair packs, hair tonics, permanent wave fixing solutions, hair coloring shampoos, hair coloring agents, hair fixing agents, hair setting agents, hair styling preparations, blow-drying lotions, styling mousses, hair gels, hair waxes or combinations thereof.

Preferred agents according to the invention are shampoos, conditioning agents or hair tonics. In a preferred embodiment of the invention an agent according to the invention can moreover also include UV filters (I). There are no general restrictions on the UV filters to be used according to the invention in terms of their structure and their physical properties. In fact all UV filters that can be used in the cosmetics sector whose absorption maximum is in the UVA (315-400 nm), UVB (280-315 nm) or UVC 280 nm) range are suitable. UV filters having an absorption maximum in the UVB range, in particular in the range from approximately 280 to approximately 300 nm, are particularly preferred.

The UV filters used according to the invention can be selected for example from substituted benzophenones, p-aminobenzoic acid esters, diphenyl acrylic acid esters, cinnamic acid esters, salicylic acid esters, benzimidazoles and o-aminobenzoic acid esters.

Examples of UV filters that can be used according to the invention are 4-aminobenzoic acid, N,N,N-trimethyl-4-(2-oxobom-3-ylidene methyl)aniline methyl sulfate, 3,3,5-trimethyl cyclohexyl salicylate (homosalate), 2-hydroxy-4-methoxybenzophenone (benzophenone-3; Uvinul®M 40, Uvasorb®MET, Neo Heliopan®BB, Eusolex®4360), 2-phenylbenzimidazole-5-sulfonic acid and potassium, sodium and triethanolamine salts thereof (phenylbenzimidazole sulfonic acid; Parsol®HS; Neo Heliopan®Hydro), 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione (butyl methoxydibenzoylmethane; Parsol®1789, Eusolex®9020), α-(2-oxobom-3-ylidene)toluene-4-sulfonic acid and salts thereof, ethoxylated 4-aminobenzoic acid ethyl ester (PEG-25 PABA; Uvinul®P 25), 4-dimethylaminobenzoic acid-2-ethylhexyl ester (octyl dimethyl PABA; Uvasorb®DMO, Escalo®507, Eusolex®6007), salicylic acid-2-ethylhexyl ester (octyl salicylate; Escalol®587, Neo Heliopan®OS, Uvinul®018), 4-methoxycinnamic acid isopentyl ester (isoamyl p-methoxycinnamate; Neo Heliopan E 1000), 4-methoxycinnamic acid-2-ethylhexyl ester (octyl methoxycinnamate; Parsol®MCX, Escalol®557, Neo Heliopan®AV), 2-hydroxy-4-methoxybenzophenone-5-sulfonic acid and the sodium salt thereof (benzophenone-4; Uvinul®MS 40; Uvasorb®S 5), 3-(4′-methylbenzylidene)-D,L-camphor (4-methylbenzylidene camphor; Parsol®5000, Eusolex®6300), 3-benzylidene camphor (3-benzylidene camphor), 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)-[2-oxoborn-3-ylidene methyl]benzyl}acrylamide, 2,4-dihydroxybenzophenone (benzophenone-1; Uvasorb®20 H, Uvinul®400), 1,1′-diphenylacrylonitrilic acid-2-ethylhexyl ester (octocrylene; Eusolex®OCR, Neo Heliopan®Type 303, Uvinul®N 539 SG), o-aminobenzoic acid menthyl ester (menthyl anthranilate; Neo Heliopan®MA), 2,2′,4,4′-tetrahydroxybenzophenone (benzophenone-2; Uvinul®D-50), 2,2′-dihydroxy-4,4′-dimethoxybenzophenone (benzophenone-6), 2,2′-dihydroxy-4,4′-dimethoxybenzophenone-5-sodium sulfonate and 2-cyano-3,3-diphenylacrylic acid-2′-ethylhexyl ester. 4-Aminobenzoic acid, N,N,N-trimethyl-4-(2-oxobom-3-ylidene methyl)aniline methyl sulfate, 3,3,5-trimethyl cyclohexyl salicylate, 2-hydroxy-4-methoxybenzophenone, 2-phenylbenzimidazole-5-sulfonic acid and potassium, sodium and triethanolamine salts thereof, 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione, α-(2-oxobom-3-ylidene)toluene-4-sulfonic acid and salts thereof, ethoxylated 4-aminobenzoic acid ethyl ester, 4-dimethylaminobenzoic acid-2-ethylhexyl ester, salicylic acid-2-ethylhexyl ester, 4-methoxycinnamic acid isopentyl ester, 4-methoxycinnamic acid-2-ethylhexyl ester, 2-hydroxy-4-methoxybenzophenone-5-sulfonic acid and the sodium salt thereof, 3-(4′-methylbenzylidene)-D,L-camphor, 3-benzylidene camphor, 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)-[2-oxobom-3-ylidene methyl]benzyl}acrylamide are preferred. Most particularly preferred according to the invention are 2-hydroxy-4-methoxybenzophenone, 2-phenylbenzimidazole-5-sulfonic acid and potassium, sodium and triethanolamine salts thereof, 1-(4-tert-butylphenyl)-3-(4-methoxyphenyl)propane-1,3-dione, 4-methoxycinnamic acid-2-ethylhexyl ester and 3-(4′-methylbenzylidene)-D,L-camphor.

UV filters whose molar extinction coefficient at the absorption maximum is above 15,000, in particular above 20,000, are preferred.

It has moreover been found that with structurally similar UV filters, the water-insoluble compound has in many cases the greater effect in the context of the teaching according to the invention as compared with water-soluble compounds that differ therefrom by one or more additional ionic groups. Within the context of the invention water-insoluble is understood to mean UV filters that dissolve in water at 20° C. by no more than 1 wt. %, in particular no more than 0.1 wt. %. These compounds should furthermore be soluble in conventional cosmetic oil components at room temperature by at least 0.1, in particular at least 1 wt. %. The use of water-insoluble UV filters can therefore be preferred according to the invention.

According to a further embodiment of the invention UV filters having a cationic group, in particular a quaternary ammonium group, are preferred.

These UV filters have the general structure U-Q.

The structural part U denotes a group that absorbs UV radiation. This group can in principle be derived from the aforementioned known UV filters that are suitable for use in the cosmetics sector by substituting a group, generally a hydrogen atom, of the UV filter with a cationic group Q, in particular having a quaternary amino function.

Compounds from which the structural part U can be derived are for example
substituted benzophenones,
p-aminobenzoic acid esters,
diphenyl acrylic acid esters,
cinnamic acid esters,
salicylic acid esters,
benzimidazoles and
o-aminobenzoic acid esters.

Structural parts U that derive from cinnamic acid amide or from N,N-dimethylaminobenzoic acid amide are preferred according to the invention.

The structural parts U can in principle be chosen such that the absorption maximum of the UV filters can lie in both the UVA range (315-400 nm) and in the UVB range (280-315 nm) or the UVC range (<280 nm). UV filters having an absorption maximum in the UVB range, in particular in the range from approximately 280 to approximately 300 nm, are particularly preferred.

Depending also on the structural part Q, the structural part U is furthermore preferably chosen such that the molar extinction coefficient of the UV filter at the absorption maximum is above 15,000, in particular above 20,000.

The structural part Q preferably includes a quaternary ammonium group as the cationic group. This quaternary ammonium group can in principle be linked directly to the structural part U, such that the structural part U is one of the four substituents of the positively charged nitrogen atom.

However, one of the four substituents at the positively charged nitrogen atom is preferably a group, in particular an alkylene group having 2 to 6 carbon atoms, that functions as a link between the structural part U and the positively charged nitrogen atom.

The group Q advantageously has the general structure —(CH₂)ₓ—N⁺R¹R²R³X⁻, in which x denotes an integer from 1 to 4, R¹ and R² independently of each other denote C₁₋₄ alkyl groups, R³ denotes a C₁₋₂₂ alkyl group or a benzyl group and X⁻ denotes a physiologically acceptable anion. In the context of this general structure x preferably denotes the number 3, R¹ and R² each denote a methyl group and R³ denotes either a methyl group or a saturated or unsaturated, linear or branched hydrocarbon chain having 8 to 22, in particular 10 to 18, carbon atoms.

Physiologically acceptable anions are for example inorganic anions such as halides, in particular chloride, bromide and fluoride, sulfate ions and phosphate ions as well as organic anions such as lactate, citrate, acetate, tartrate, methosulfate and tosylate.

Two preferred UV filters having cationic groups are the compounds cinnamic acid amidopropyl trimethylammonium chloride (Incroquat®UV-283) and dodecyl dimethylaminobenzamidopropyl dimethylammonium tosylate (Escalol® HP 610), which are available as commercial products.

In summary, the following UV filters are most particularly preferred: 2-hydroxy-4-methoxybenzophenone (benzophenone-3; Uvinul®M 40, Uvasorb®MET, Neo Heliopan®BB, Eusolex®4360), 2-phenylbenzimidazole-5-sulfonic acid and the potassium, sodium and triethanolamine salts thereof (phenylbenzimidazole sulfonic acid; Parsol®HS; Neo Heliopan®Hydro), 3,3′-(1,4-phenylenedimethylene)bis(7,7-dimethyl-2-oxobicyclo-[2.2.1]hept-1-yl methanesulfonic acid) and salts thereof, 1-(4-tert-butylphenyl)-3-(4-methoxyphenyl)propane-1,3-dione (butyl methoxydibenzoylmethane; Parsol®1789, Eusolex®9020), α-(2-oxoborn-3-ylidene)toluene-4-sulfonic acid and salts thereof, ethoxylated 4-aminobenzoic acid ethyl ester (PEG-25 PABA; Uvinul®P 25), 4-dimethylaminobenzoic acid-2-ethylhexyl ester (octyl dimethyl PABA; Uvasorb®DMO, Escalol® 507, Eusolex®6007), salicylic acid-2-ethylhexyl ester (octyl salicylate; Escalol®587, Neo Heliopan®OS, Uvinul®O18), 4-methoxycinnamic acid isopentyl ester (isoamyl p-methoxycinnamate; Neo Heliopan®E 1000), 4-methoxycinnamic acid-2-ethylhexy ester (octyl methoxycinnamate; Parsol®MCX, Escalol®557, Neo Heliopan®AV), 2-hydroxy-4-methoxybenzophenone-5-sulfonic acid and the sodium salt thereof (benzophenone-4; Uvinul®MS 40; Uvasorb®S 5), 2,2′,4,4′-tetrahydroxybenzophenone (benzophenone-2; Uvinul D-50), 2,2′-dihydroxy-4,4′-dimethoxybenzophenone (benzophenone-6), cinnamic acid amidopropyl trimethylammonium chloride (Incroquat UV-283) and dodecyl dimethylaminobenzamidopropyl dimethylammonium tosylate (Escalol® HP 610).

Of these, the following UV filters are highly preferred: benzophenone-3, benzophenone-4, octyl methoxycinnamate, benzophenone-6, cinnamic acid amidopropyl trimethylammonium chloride and dodecyl dimethylaminobenzamidopropyl dimethylammonium tosylate.

Finally, the UV filters according to the invention also include inorganic substances such as titanium dioxide and zinc oxide. These crystalline structures are preferably used in the form of nanoparticles. To achieve a high UV protection factor it can be particularly advantageous if in particular titanium dioxide is used in addition to the organic UV protection filters. A high UV protection factor is understood to be a factor of 20 and higher, preferably 30 and higher. In the case of hair treatment agents intended in particular for infants, a UV protection factor of 50 and higher should be achieved. In this case it is most particularly preferable additionally to use titanium dioxide in particular.

The teaching according to the invention naturally also comprises the use of a combination of a plurality of UV filters. In the context of this embodiment the combination of at least one water-insoluble UV filter with at least one UV filter having a cationic group is preferred. If at least two or more UV filters are used, the UV filters are used in equal amounts.

The UV filters (I) are conventionally included in the agents according to the invention in a total amount from 0.1 to 20.0 wt. %, relative to the total agent. Amounts from 0.1 to 15.0 wt. % are preferred, with amounts from 0.2 to 10.0 wt. % being most preferred.

As the second essential ingredient the agents according to the invention include 0.1 to 30 wt. % of at least one compound of the general formula (I). In preferred agents according to the invention the compounds of formula (I) are used within relatively narrow quantity ranges, such that preferred hair treatment agents according to the invention are characterized in that they include 0.25 to 25 wt. %, preferably 0.5 to 20 wt. %, more preferably 1 to 15 wt. %, still more preferably 1.5 to 10 wt. % and in particular 2 to 5 wt. % of at least one compound of the general formula (I).

The compounds of formula (I) in which n=m=20 are preferred in particular.

The residues R and R′ are selected independently of one another from —H or —CH₃. In preferred compounds of formula (I) R=such that either PEG or PPG diesterquats are used. Most particularly preferably, R═R′═—CH₃.

Halides such as chloride, bromide or iodide are suitable as the physiologically acceptable anion X⁻, but also toluene sulfonate, methosulfate, etc. A particularly preferred anion is methosulfate, such that preferred hair treatment agents according to the invention include 0.25 to 25 wt. %, preferably 0.5 to 20 wt. %, more preferably 1 to 15 wt. %, still more preferably 1.5 to 10 wt. % and in particular 2 to 5 wt. % of at least one compound of the general formula (Ia)

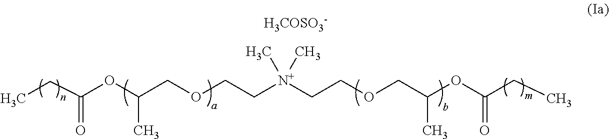

in which
n and m independently of one another denote integers between 5 and 40, with the proviso that n+m≧38;
a and b independently of one another denote 1, 2, 3, 4 or 5.

All that has been stated above in respect of indices n and m also applies to formula (Ia). A compound of formula (Ia) that is preferred in particular can thus be described by formula (Ia-1):

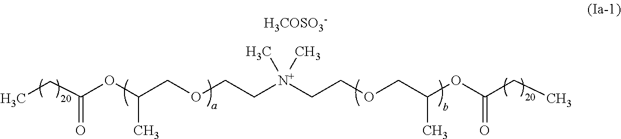

Indices a and b preferably independently of one another denote 1, 2, 3, 4 or 5. More preferably the equation a+2≧b≧a−2 applies here. Most particularly preferably a=b, such that the five compounds with a=b=1 or a=b=2 or a=b=3 or a=b=4 or a=b=5 are particularly preferred.

Compounds of formula (Ia-1) where a=b=3, represented below as formula (Ib), are preferred in particular, as they deliver particularly pronounced effects in the combination according to the invention. Hair treatment agents that are preferred according to the invention are therefore characterized in that they include 0.5 to 20 wt. %, preferably 0.75 to 15 wt. %, more preferably 1 to 10 wt. %, still more preferably 1.5 to 7.5 wt. % and in particular 2 to 4.5 wt. % of at least one compound of the general formula (Ib)

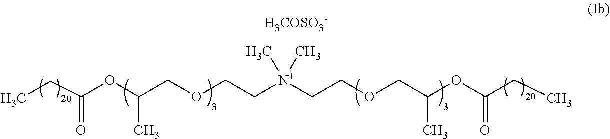

It has been found that the care effects of the agents according to the invention can be further increased and in particular the stability of the agents further improved if the agents include certain acylated diamines in addition to the compound(s) of formula (I).

Preferred hair treatment agents according to the invention are therefore characterized in that they additionally include 0.1 to 10 wt. % of at least one compound of formula (II)

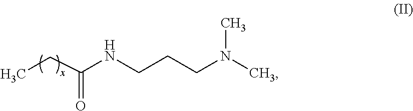

in which x denotes 18, 19, 20, 21, 22, 23 or 24.

Compounds of formula (II) with n=20 are particularly preferred.

The care effects of the agents according to the invention can be further strengthened by the use of certain care substances. These are preferably selected from certain groups of care substances known per se, since in terms of their formulation and their care effect these care substances harmonize superbly with the combination used according to the invention.

Hair treatment agents that are preferred according to the invention are characterized in that they additionally include care substance(s)—relative to their weight—in amounts from 0.001 to 10 wt. %, preferably 0.005 to 7.5 wt. %, particularly preferably 0.01 to 5 wt. % and in particular 0.05 to 2.5 wt. %, preferred care substance(s) being selected from the group comprising

i. L-carnitine and/or salts thereof;
ii. taurine and/or salts thereof;
iii. niacinamide;
iv. ubiquinone;
v. ectoine.

L-Carnitine (IUPAC name (R)-(3-Carboxy-2-hydroxypropyl)-N,N,N-trimethylammonium hydroxide), is a naturally occurring, vitamin-like substance.

As a betaine, L-carnitine can form addition compounds and double salts. L-Carnitine derivatives that are preferred according to the invention are selected in particular from acetyl L-carnitine, L-carnitine fumarate, L-carnitine citrate, lauroyl L-carnitine and particularly preferably L-carnitine tartrate. The specified L-carnitine compounds are available for example from Lonza GmbH (Wuppertal, Germany).

Preferred hair treatment agents according to the invention are characterized in that they include—relative to their weight—0.001 to 10 wt. %, preferably 0.005 to 7.5 wt. %, particularly preferably 0.01 to 5 wt. % and in particular 0.05 to 2.5 wt. % of L-carnitine or L-carnitine derivatives, preferred L-carnitine derivatives being selected from acetyl L-carnitine, L-carnitine fumarate, L-carnitine citrate, lauroyl L-carnitine and in particular L-carnitine tartrate.

A further preferred care substance which can be used, and which has activating properties, is taurine. Hair treatment agents that are preferred according to the invention include—relative to their weight—0.01 to 15 wt. %, preferably 0.025 to 12.5 wt. %, particularly preferably 0.05 to 10 wt. %, more preferably 0.1 to 7.5 wt. % and in particular 0.5 to 5 wt. % of taurine (2-aminoethanesulfonic acid).

A further preferred group of care substances in the agents according to the invention are vitamins, provitamins or vitamin precursors. These are described below:

The group of substances classed as vitamin A includes retinol (vitamin A₁) and 3,4-didehydroretinol (vitamin A₂). β-Carotene is the retinol provitamin. Suitable vitamin A components according to the invention are for example vitamin A acid and esters thereof, vitamin A aldehyde and vitamin A alcohol and esters thereof such as the palmitate and acetate. The agents according to the invention include the vitamin A component preferably in amounts from 0.05 to 1 wt. %, relative to the total preparation.

The vitamin B group or vitamin B complex includes inter alia
Vitamin B₁ (thiamine)
Vitamin B₂ (riboflavin)
Vitamin B₃. The compounds nicotinic acid and nicotinic acid amide (niacinamide) are often included under this term. Preferred according to the invention is nicotinic acid amide, which is preferably included in the agents used according to the invention in amounts from 0.05 to 1 wt. %, relative to the total agent.
Vitamin B₅ (pantothenic acid, panthenol and pantolactone). Within the context of this group panthenol and/or pantolactone is preferably used (see below). Derivatives of panthenol which can be used according to the invention are in particular the esters and ethers of panthenol as well as cationically derivatized panthenols. Individual representatives are for example panthenol triacetate, panthenol monoethyl ether and the monoacetate thereof as well as the cationic panthenol derivatives disclosed in WO 92/13829. The cited compounds of the vitamin B₅ type are preferably included in the agents according to the invention in amounts from 0.05 to 10 wt. %, relative to the total agent. Amounts from 0.1 to 5 wt. % are particularly preferred.
Vitamin B₆ (pyridoxine as well as pyridoxamine and pyridoxal).

Vitamin C (ascorbic acid). Vitamin C is used in the agents according to the invention preferably in amounts from 0.1 to 3 wt. %, relative to the total agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof, which include in particular esters such as acetate, nicotinate, phosphate and succinate, are preferably included in the agents according to the invention in amounts from 0.05 to 1 wt. %, relative to the total agent.

Vitamin F. The term “vitamin F” is conventionally understood to mean essential fatty acids, in particular linoleic acid, linolenic acid and arachidonic acid.

Vitamin H. Vitamin H is the name given to the compound (3aS,4S,6aR)-2-oxohexahydrothienol[3,4-d]-imidazole-4-valeric acid, although this is now more widely known by the trivial name biotin. Biotin is preferably included in the agents according to the invention in amounts from 0.0001 to 1.0 wt. %, in particular in amounts from 0.001 to 0.01 wt. %.

In summary, hair treatment agents according to the invention are preferred which include—relative to their weight—0.1 to 5 wt. %, preferably 0.2 to 4 wt. %, particularly preferably 0.25 to 3.5 wt. %, more preferably 0.5 to 3 wt. % and in particular 0.5 to 2.5 wt. % of vitamins and/or provitamins and/or vitamin precursors, which are preferably assigned to groups A, B, C, E, F and H, wherein preferred agents include-2,4-dihydroxy-N-(3-hydroxypropyl)-3,3-dimethylbutyramide, provitamin B₅) and/or pantothenic acid (vitamin B3, vitamin B5) and/or niacin, niacinamide or nicotinamide (vitamin B₃) and/or L-ascorbic acid (vitamin C) and/or thiamine (vitamin B₁) and/or riboflavin (vitamin B2, vitamin G) and/or biotin (vitamin B7, vitamin H) and/or folic acid (vitamin B9, vitamin B_{c} or vitamin M) and/or vitamin B₆ and/or vitamin B₁₂.

It has been found that certain quinones are particularly suitable as a care substance. As a further care substance the agents according to the invention can therefore include 0.0001 to 5 wt. % of at least one bioquinone of the formula (Ubi)

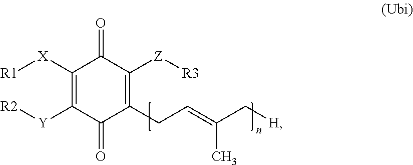

in which
X, Y, Z independently of one another denote —O— or —NH— or NR⁴— or a chemical bond
R¹, R², R³ independently of one another denote a hydrogen atom or an optionally substituted aryl group or an optionally substituted (C₁-C₆)alkyl group or a hydroxyalkyl group or a polyhydroxyalkyl group or an optionally substituted (C₁-C₆)alkylene group, or a (C₁-C₆) acyl residue, wherein preferred residues are selected independently of one another from —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃
R⁴ denotes —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃
n denotes values from 1 to 20, preferably from 2 to 15, and in particular 5, 6, 7, 8, 9, 10.

Particularly preferred hair treatment agents according to the invention are characterized in that as a care substance they include—relative to their weight—0.0001 to 1 wt. %, preferably 0.001 to 0.5 wt. % and particularly preferably 0.005 to 0.1 wt. % of at least one ubiquinone and/or at least one ubiquinol and/or at least one derivative of these substances, wherein preferred agents include a ubiquinone of formula (Ubi)

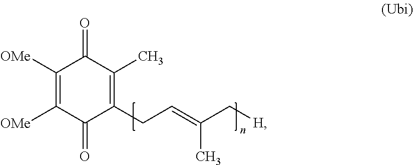

in which n denotes the values 6, 7, 8, 9 or 10, particularly preferably 10 (coenzyme Q10).

As an alternative to or in addition to the particularly preferred ubiquinones, the agents according to the invention can also include plastoquinones. Preferred agents according to the invention are characterized in that they include 0.0002 to 4 wt. %, preferably 0.0005 to 3 wt. %, particularly preferably 0.001 to 2 wt. %, more preferably 0.0015 to 1 and in particular 0.002 to 0.5 wt. % of at least one plastoquinone of the formula (Ubi-b)

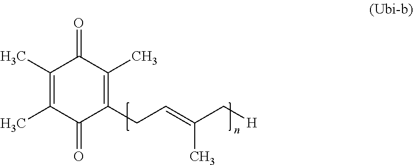

in which n denotes values from 1 to 20, preferably from 2 to 15, and in particular 5, 6, 7, 8, 9, 10.

As a further care enhancer the agents according to the invention can include ectoine. Ectoine ((4S)-2-methyl-1,4,5,6-tetrahydropyrimidine-4-carboxylic acid) is a natural substance belonging to the group of compatible solutes. The highly water-binding, low-molecular-weight organic compound occurs in halophilic bacteria and allows these extremophile organisms to survive under stress conditions. Hair treatment agents that are preferred according to the invention are characterized in that they include—relative to their weight—0.001 to 10 wt. %, preferably 0.01 to 5 wt. %, particularly preferably 0.05 to 2.5 wt. % and in particular 0.1 to 1 wt. % of (S)-2-methyl-1,4,5,6-tetrahydro-4-pyrimidinecarboxylic acid (ectoine) and the physiologically acceptable salts of this compound and/or (S,S)-5-hydroxy-2-methyl-1,4,5,6-tetrahydro-4-pyrimidinecarboxylic acid (hydroxyectoine) and the physiologically acceptable salts of this compound.

To improve the elasticity and strength of the internal structure of hair treated with agents according to the invention, the agents according to the invention can include purine and/or purine derivatives as a care substance. In particular, the combination of purine and/or purine derivatives with ubiquinones and/or plastoquinones as a care substance means that hair treated with corresponding agents exhibits inter alia higher measured values in differential thermal analysis and improved wet and dry combability.

Purine (7H-imidazo[4,5-d]pyrimidine) does not occur freely in nature but forms the parent substance of purines. Purines for their part are a group of important compounds that occur widely in nature and are involved in human, animal, plant and microbial metabolic processes; they are derived from the parent substance by substitution with OH, NH₂, SH in the 2-, 6- and 8-position and/or with CH₃ in the 1-, 3-, 7-position. Purine can be produced for example from aminoacetonitrile and formamide. Purines and purine derivatives are often isolated from natural substances but are also available synthetically via many routes.

Preferred agents according to the invention include purine and/or purine derivatives in relatively narrow quantity ranges. Cosmetic agents that are preferred according to the invention are characterized in that they include—relative to their weight—0.001 to 2.5 wt. %, preferably 0.0025 to 1 wt. %, particularly preferably 0.005 to 0.5 wt. % and in particular 0.01 to 0.1 wt. % of purine(s) and/or purine derivative(s).

Some representatives of purine, purines and purine derivatives are particularly preferred according to the invention. Hair treatment agents that are preferred according to the invention are characterized in that as a care substance they include—relative to their weight—0.001 to 2.5 wt. %, preferably 0.0025 to 1 wt. %, particularly preferably 0.005 to 0.5 wt. % and in particular 0.01 to 0.1 wt. % of purine(s) and/or purine derivative(s), wherein preferred agents include purine and/or purine derivative(s) of the formula (Pur-I)

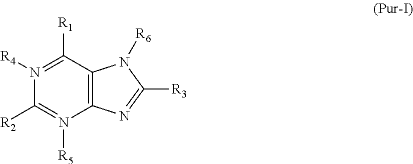

in which the residues R¹, R² and R³ are selected independently of one another from —H, —OH, NH₂, —SH and the residues R⁴, R⁵ and R⁶ are selected independently of one another from —H, —CH₃ and —CH₂—CH₃, the following compounds being preferred:
purine (R¹═R²═R³═R⁴═R⁵═R⁶═H), adenine (R¹═NH₂, R²═R³═R⁴═R⁵═R⁶═H), guanine (R¹═OH, R²═NH₂, R³═R⁴═R⁵═R⁶═H), uric acid (R¹═R²═R³═OH, R⁴═R⁵═R⁶═H), hypoxanthine (R¹═OH, R²═R³═R⁴═R⁵═R⁶H), 6-purinethiol (R¹═SH, R²═R³═R⁴═R⁵═R⁶═H), 6-thioguanine (R¹═SH, R²═NH₂, R³═R⁴═R⁵═R⁶═H), xanthine (R¹═R²═OH, R³═R⁴═R⁵═R⁶═H), caffeine (R¹═R²═OH, R³═H, R⁴═R⁵═R⁶═CH₃), theobromine (R¹═R²═OH, R³═R⁴═H, R⁵═R⁶═CH₃), theophylline (R¹═R²═OH, R³═H, R⁴═CH₃, R⁵═CH₃, R⁶═H).

It is furthermore advantageous to use purine or purine derivatives and bioquinones in a defined ratio to one another. Agents according to the invention are preferred here in which the weight ratio of purine (derivative(s)) and bioquinone(s) is 10:1 to 1:100, preferably 5:1 to 1:50, particularly preferably 2:1 to 1:20 and in particular 1:1 to 1:10.

As has already been mentioned, caffeine is a particularly preferred purine derivative, and coenzyme Q10 is a particularly preferred bioquinone. Particularly preferred agents according to the invention are therefore characterized in that they include—relative to their weight—0.001 to 2.5 wt. %, preferably 0.0025 to 1 wt. %, particularly preferably 0.005 to 0.5 wt.-% and in particular 0.01 to 0.1 wt. % of caffeine and 0.0002 to 4 wt. %, preferably 0.0005 to 3 wt. %, particularly preferably 0.001 to 2 wt. %, more preferably 0.0015 to 1 and in particular 0.002 to 0.5 wt. % of coenzyme Q10.

The agents according to the invention can also include flavonoids as a care substance. The flavonoids are a group of water-soluble plant dyes and they play an important role in the metabolism of many plants. Along with phenolic acids they belong to the polyphenols. There are well over 6500 different flavonoids known, and they can be divided into flavonols, flavones, flavanones, isoflavonoids and anthocyanins.

Flavonoids from all six groups can be used according to the invention, with certain representatives from the individual groups being preferred as a care substance because of their particularly intensive action. Preferred flavonols are quercetin, rutin, kaempferol, myricetin, isorhamnetin, preferred flavanols are catechin, gallocatechin, epicatechin, epigallocatechin gallate, theaflavin, thearubigin, preferred flavones are luteolin, apigenin, morin, preferred flavanones are hesperetin, naringenin, eriodictyol, preferred isoflavonoids are genistein, daidzein, and preferred anthocyanidins (anthocyanins) are cyanidin, delphinidin, malvidin, pelargonidin, peonidin, petunidin.

Hair treatment agents that are particularly preferred according to the invention are characterized in that they include—relative to their weight—0.001 to 2.5 wt. %, preferably 0.0025 to 1 wt. %, particularly preferably 0.005 to 0.5 wt. % and in particular 0.01 to 0.1 wt. % of flavonoids, in particular flavonols, particularly preferably 3,3′,4′,5,7-pentahydroxyflavone (quercetin) and/or 3,3′,4′,5,7-pentahydroxyflavone-3-O-rutinoside (rutin).

Also preferred is the use of bisabolol and/or bisabolol oxides as a care substance in the agents according to the invention. Hair treatment agents according to the invention are preferred here which additionally include 0.001 to 5 wt. %, preferably 0.01 to 4 wt. %, particularly preferably 0.02 to 2.5 wt. % and in particular 0.1 to 1.5 wt. % of bisabolol and/or oxides of bisabolol, preferably (−)-alpha-bisabolol

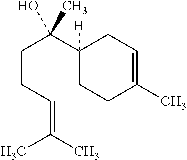 [SMILES: CC(C)=CCC[C@](C)(O)[C@H]1CC=C(C)CC1]

Creatine is also suitable according to the invention as a care substance. Creatine (3-methyl guanidinoacetic acid) is an organic acid which in vertebrates helps inter alia to supply energy to the muscles. Creatine is synthesized in the kidneys, the liver and the pancreas. It is formally derived from the amino acids glycine and arginine and 95% of it is present in the skeletal muscle. Particularly preferred hair treatment agents according to the invention include—relative to their weight—0.01 to 15 wt. %, preferably 0.025 to 12.5 wt. %, particularly preferably 0.05 to 10 wt. %, more preferably 0.1 to 7.5 wt. % and in particular 0.5 to 5 wt. % of N-methylguanidinoacetic acid (creatine).

The agents according to the invention can include in addition to the aforementioned ingredients and optional further ingredients further substances which prevent, alleviate or cure hair loss. A content of active ingredients which stabilize the hair root is advantageous in particular. These substances are described below:

Propecia (finasteride) is currently the only preparation that is approved worldwide and for which an effectiveness and tolerance has been proven in numerous studies. Propecia works by reducing the ability of DHT to form from testosterone.

Minoxidil with or without supplementary additives is probably the oldest demonstrably effective hair growth agent. For the treatment of hair loss it should be used for external application only. There are hair lotions including 2% to 5% minoxidil, also gels including up to 15% minoxidil. The effectiveness increases with the dose, but in hair lotions minoxidil is soluble only in a proportion of up to 5%. In many countries hair lotions including up to 2% minoxidil are available without a prescription.

Spironolactone in the form of a hair lotion and in combination with minoxidil can be used for external application to combat hormonal influences on the hair follicles. Spironolactone works as an androgen receptor blocker, in other words binding of DHT to the hair follicles is prevented.

In summary, hair treatment agents according to the invention are preferred which additionally include—relative to their weight—0.001 to 5 wt. % of hair root-stabilizing substances, in particular minoxidil and/or finasteride and/or ketoconazole.

In addition to the care substances the agents according to the invention can include further care substances. Their presence is not absolutely essential for achieving the effects according to the invention, but advanced effects, such as a pleasant feel or a pleasant application texture, can result from the use of these care substances.

In summary, hair treatment agents according to the invention are preferred which include 0.15 to 15 wt. %, preferably 0.2 to 10 wt. %, more preferably 0.25 to 7.5 wt. %, still more preferably 0.5 to 5 wt. % and in particular 0.75 to 2.5 wt. % of cosmetic oil(s) from the groups of
vegetable oils,
liquid paraffin oils, isoparaffin oils and synthetic hydrocarbons as well as di-n-alkyl ethers,
esters of C₆-C₃₀ fatty acids with C₂-C₃₀ fatty alcohols,
dicarboxylic acid esters,
symmetrical, asymmetrical or cyclic esters of carbonic acid with fatty alcohols,
tri-fatty acid esters of saturated and/or unsaturated linear and/or branched fatty acids with glycerol,
fatty acid partial glycerides,
silicone(s).

As the first essential ingredient the agents according to the invention include 0.1 to 20 wt. % of at least one cosmetic oil. These oil bodies preferably have a melting point below 50° C., more preferably below 45° C., particularly preferably below 40° C., highly preferably below 35° C. and most preferably the cosmetic oils are free-flowing at a temperature below 30° C. Preferred cosmetic oils are defined and described in more detail below.

The natural and synthetic cosmetic oils include, for example:
vegetable oils. Examples of such oils are sunflower oil, olive oil, soybean oil, rapeseed oil, almond oil, jojoba oil, orange oil, wheat germ oil, peach kernel oil and the liquid components of coconut oil. Other triglyceride oils are also suitable, however, such as the liquid components of beef fat and synthetic triglyceride oils
liquid paraffin oils, isoparaffin oils and synthetic hydrocarbons and also di-n-alkyl ethers having in total between 12 and 36 C atoms, in particular between 12 and 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. The compounds available as commercial products, 1,3-di-(2-ethylhexyl)cyclohexane (Cetiol® S) and di-n-octyl ether (Cetiol® OE), can be preferred.
silicones. These preferably come from the groups of dimethicones and/or cyclomethicones and/or amodimethicones and/or dimethiconols and/or trisiloxanes.
ester oils. Ester oils are understood to be the esters of C₆-C₃₀ fatty acids with C₂-C₃₀ fatty alcohols. The monoesters of fatty acids with alcohols having 2 to 24 C atoms are preferred. Examples of fatty acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Examples of the fatty alcohol components in the ester oils are isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. Particularly preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate,
symmetrical, asymmetrical or cyclic esters of carbonic acid with fatty alcohols, glycerol carbonate or dicaprylyl carbonate (Cetiol® CC),
tri-fatty acid esters of saturated and/or unsaturated linear and/or branched fatty acids with glycerol,
fatty acid partial glycerides, namely monoglycerides, diglycerides and technical mixtures thereof. If technical products are used, small amounts of triglycerides may also be included for production reasons. The partial glycerides preferably obey formula (D4-I),

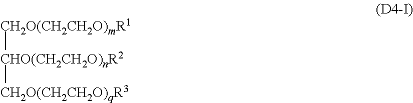

in which R¹, R² and R³ independently of one another denote hydrogen or a linear or branched, saturated and/or unsaturated acyl residue having 6 to 22, preferably 12 to 18, carbon atoms, with the proviso that at least one of these groups denotes an acyl residue and at least one of these groups denotes hydrogen. The sum (m+n+q) denotes 0 or numbers from 1 to 100, preferably 0 or 5 to 25. R¹ preferably denotes an acyl residue and R² and R³ hydrogen and the sum (m+n+q) is preferably O. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Oleic acid monoglycerides are preferably used.

Preferred cosmetic oils are vegetable oils. Suitable natural oils are for example amaranth seed oil, apricot kernel oil, argan oil, avocado oil, babassu oil, cottonseed oil, borage seed oil, camelina oil, thistle oil, groundnut oil, pomegranate kernel oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, palm oil, rapeseed oil, rice oil, sea buckthorn fruit oil, sea buckthorn seed oil, sesame oil, shea butter, soybean oil, sunflower oil, grape seed oil, walnut oil or wild rose oil.

Preferred natural oils include at least one of the fatty acids palmitic acid, stearic acid and linoleic acid. Particularly preferred natural oils include the fatty acids palmitic acid, stearic acid and linoleic acid in a total amount of at least 50 wt. % of the fatty acids. Most particularly preferred oils are moreover characterized by an additional content of squalene. Highly preferred natural oils and mixtures thereof also have a proportion of linolenic acids.

The teaching according to the invention naturally also comprises the fact that at least two natural oils can be mixed together. Preferred mixtures of natural oils are amaranth seed oil with at least one sea buckthorn oil, amaranth seed oil with shea butter, amaranth seed oil with camelina oil, amaranth seed oil with olive oil, amaranth seed oil with macadamia nut oil, olive oil with at least one sea buckthorn oil, olive oil with camelina oil, olive oil with shea butter, macadamia nut oil and at least one sea buckthorn oil, macadamia nut oil with shea butter.

Argan oil is one of the particularly preferred natural oils. A further preferred natural oil is amaranth seed oil. An oil that is suitable according to the invention is available for example under the trade name Amaranth Seed Oil from Euro Ingredients. Shea butter is a further example of the natural oils.

In summary, hair treatment agents that are preferred according to the invention are characterized in that they include 0.15 to 15 wt. %, preferably 0.2 to 10 wt. %, more preferably 0.25 to 7.5 wt. %, still more preferably 0.5 to 5 wt. % and in particular 0.75 to 2.5 wt. % of at least one vegetable oil from the group comprising amaranth seed oil, apricot kernel oil, argan oil, avocado oil, babassu oil, cottonseed oil, borage seed oil, camelina oil, thistle oil, groundnut oil, pomegranate kernel oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, palm oil, rapeseed oil, rice oil, sea buckthorn fruit oil, sea buckthorn seed oil, sesame oil, shea butter, soybean oil, sunflower oil, grape seed oil, walnut oil or wild rose oil.

Ester oils are another preferred group of cosmetic oils. Particularly preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

In summary, hair treatment agents that are preferred according to the invention are characterized in that they include 0.15 to 15 wt. %, preferably 0.2 to 10 wt. %, more preferably 0.25 to 7.5 wt. %, still more preferably 0.5 to 5 wt. % and in particular 0.75 to 2.5 wt. % of at least one ester of acids from the group comprising hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof with at least one alcohol from the group comprising isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof, wherein isopropyl myristate, isononanoic acid C16-18 alkyl ester, 2-ethylhexyl palmitate, stearic acid-2-ethylhexyl ester, cetyl oleate, glycerol tricaprylate, coconut fatty alcohol caprinate/caprylate, n-butyl stearate, oleyl erucate, isopropyl palmitate, oleyl oleate, lauric acid hexyl ester, di-n-butyl adipate, myristyl myristate, cetearyl isononanoate, oleic acid decyl ester are preferred.

Further preferred cosmetic oils are symmetrical, asymmetrical or cyclic esters of carbonic acid with fatty alcohols. Hair treatment agents that are preferred according to the invention are characterized in that they include 0.15 to 15 wt. %, preferably 0.2 to 10 wt. %, more preferably 0.25 to 7.5 wt. %, still more preferably 0.5 to 5 wt. % and in particular 0.75 to 2.5 wt. % of at least one ester of carbonic acid from the group of glycerol carbonate and/or dicaprylyl carbonate.

Silicones are another preferred group of cosmetic oils. The silicones preferably come from the groups of dimethicones and/or cyclomethicones and/or amodimethicones and/or dimethiconols and/or trisiloxanes.

Dimethicones can be both linear and branched and also cyclic or cyclic and branched. Linear dimethicones can be represented by the following structural formula (Si1):

(SiR¹₃)—O—(SiR²₂—O—)ₓ—(SiR¹₃) (Si1)

Branched dimethicones can be represented by the structural formula (Si1.1):

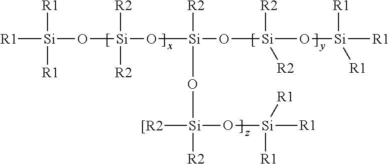

Residues R¹ and R² independently of one another denote hydrogen, a methyl residue, a C2 to C30 linear, saturated or unsaturated hydrocarbon residue, a phenyl residue and/or an aryl residue. Non-restricting examples of the residues represented by R¹ and R² include alkyl residues such as methyl, ethyl, propyl, isopropyl, butyl, isobutyl, pentyl, isopentyl, neopentyl, amyl, isoamyl, hexyl, isohexyl and similar; alkenyl residues, such as vinyl, halovinyl, alkyl vinyl, allyl, haloallyl, alkyl allyl; cycloalkyl residues, such as cyclobutyl, cyclopentyl, cyclohexyl and similar; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and similar, and sulfur-including residues, such as mercaptoethyl, mercaptopropyl, mercaptohexyl, mercaptophenyl and similar; R¹ and R² are preferably an alkyl residue including 1 to approximately 6 carbon atoms, and R¹ and R² are most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—; and —(CH₂)₃C(O)SCH₂CH₂—. Methyl, phenyl and C2 to C22 alkyl residues are preferred as R¹ and R². Of the C2 to C22 alkyl residues, lauryl, stearyl and behenyl residues are most particularly preferred. The numbers x, y and z are integers and each run independently of one another from 0 to 50,000. The molecular weights of the dimethicones are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, most particularly preferred viscosities are between 10,000 and 3,000,000 cPs. The most preferred range is between 50,000 and 2,000,000 cPs. Viscosities around the range of approximately 60,000 cPs are highly preferred. The word “approximately” defines a deviation from the stated value following the word “approximately” that is considered usual by the person skilled in the art in connection with products manufactured in industry. Reference is made here by way of example to the product “Dow Corning 200 with 60,000 cSt”.

The teaching according to the invention naturally also comprises the fact that the dimethicones can already be in the form of an emulsion.

If dimethicones are used as an emulsion, then according to the invention the droplet size of the emulsified particles is 0.01 μm to 10,000 μm, preferably 0.01 to 100 μm, most particularly preferably 0.01 to 20 μm and most preferably 0.01 to 10 μm. The particle size is determined by the light scattering method.

Particularly preferred hair treatment agents according to the invention are characterized in that they include at least one silicone of formula (Si1.2)

(CH₃)₃Si—[O—Si(CH₃)₂]ₓ—O—Si(CH₃)₃ (Si1.2),

in which x denotes a number from 0 to 100, preferably from 0 to 50, more preferably from 0 to 20 and in particular 0 to 10.

The dimethicones (Si1) are included in the compositions according to the invention by preference in amounts from 0.01 to 10 wt. %, preferably 0.01 to 8 wt. %, particularly preferably 0.1 to 7.5 wt. % and in particular 0.1 to 5 wt. %, relative to the total composition.

Particularly preferred agents according to the invention include one or more amino-functional silicones. Such silicones can be described for example by the formula (Si-2)

M(RₐQ_{b}SiO_{(4-a-b)/2})ₓ(R_{c}SiO_{(4-c)/2})_{y}M (Si-2)

wherein in the above formula
R is a hydrocarbon or a hydrocarbon residue having 1 to approximately 6 carbon atoms,
Q is a polar residue of the general formula —R¹HZ,
   in which
   R¹ is a divalent linking group that is bonded to hydrogen and to the residue Z, composed of carbon and hydrogen atoms, carbon, hydrogen and oxygen atoms or carbon, hydrogen and nitrogen atoms, and
   Z is an organic, amino-functional residue including at least one amino-functional group;
a assumes values in the range from approximately 0 to approximately 2,
b assumes values in the range from approximately 1 to approximately 3,
a+b is less than or equal to 3, and
c is a number in the range from approximately 1 to approximately 3, and
x is a number in the range from 1 to approximately 2000, preferably from approximately 3 to approximately 50 and most preferably from approximately 3 to approximately 25, and
y is a number in the range from approximately 20 to approximately 10,000, preferably from approximately 125 to approximately 10,000 and most preferably from approximately 150 to approximately 1000, and
M is a suitable silicone end group as is known in the prior art, preferably trimethylsiloxy.

Non-restricting examples of the residues represented in formula (Si-2) by R include alkyl residues such as methyl, ethyl, propyl, isopropyl, isopropyl, butyl, isobutyl, amyl, isoamyl, hexyl, isohexyl and similar; alkenyl residues, such as vinyl, halovinyl, alkyl vinyl, allyl, haloallyl, alkyl allyl; cycloalkyl residues, such as cyclobutyl, cyclopentyl, cyclohexyl and similar; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and similar, and sulfur-including residues, such as mercaptoethyl, mercaptopropyl, mercaptohexyl, mercaptophenyl and similar; R is preferably an alkyl residue including 1 to approximately 6 carbon atoms, and R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(O)OCH₂—, —(CH₂)₃ CC(O)OCH₂CH₂—, —C₆H₄C₆H₄—, —C₆H₄—CH₂C₆H₄—; and —(CH₂)₃C(O)SCH₂CH₂—.

According to formula (Si-2) Z is an organic, amino-functional residue including at least one functional amino group. A possible formula for said Z is NH(CH₂)_{z}NH₂, in which z is an integer greater than or equal to 1. Another possible formula for said Z is —NH(CH₂)_{z}(CH₂)_{zz}NH, in which both z and zz are independently of each other an integer greater than or equal to 1, this structure encompassing diamino ring structures, such as piperazinyl. Said Z is most preferably an —NHCH₂CH₂NH₂ residue. Another possible formula for said Z is —N(CH₂)_{z}(CH₂)_{zz}NX₂ or —NX₂, in which each X of X₂ is selected independently from the group consisting of hydrogen and alkyl groups having 1 to 12 carbon atoms, and zz is 0.

Q according to formula (Si-2) is most preferably a polar amino-functional residue of the formula —CH₂CH₂CH₂NHCH₂CH₂NH₂.

In formula (Si-2) a assumes values in the range from 0 to 2, b assumes values in the range from 2 to 3, a+b is less than or equal to 3, and c is a number in the range from 1 to 3. The molar ratio of the RₐQ_{b}SiO_{(4-a-b)/2} units to the R_{c}SiO_{(4-c)/2} units is in the range from approximately 1:2 to 1:65, preferably from approximately 1:5 to approximately 1:65 and most preferably from approximately 1:15 to approximately 1:20. If one or more silicones of the above formula (Si-2) is used, then the different variable substituents in the above formula can differ in the different silicone components that are present in the silicone mixture.

Preferred hair treatment agents according to the invention include an amino-functional silicone of formula (Si-3)

R′ₐG₃₋ₐ—Si(OSiG₂)ₙ—(OSiG_{b}R′_{2-b})ₘ—O—SiG₃₋ₐ-R′ₐ (Si-3),

in which:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a denotes a number between 0 and 3, in particular 0;
b denotes a number between 0 and 1, in particular 1;
m and n are numbers whose sum (m+n) is between 1 and 2000, preferably between 50 and 150, wherein n preferably assumes values from 0 to 1999 and in particular from 49 to 149, and m preferably assumes values from 1 to 2000, in particular from 1 to 10;
R′ is a monovalent residue selected from
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻,
      in which each Q denotes a chemical bond, —CH₂—, —CH₂—CH₂—, —CH₂CH₂CH₂—, —C(CH₃)₂—, —CH₂CH₂CH₂CH₂—, —CH₂C(CH₃)₂—, —CH(CH₃)CH₂CH₂—,
      R″ denotes identical or different residues from the group —H, -phenyl, -benzyl, —CH₂—CH(CH₃)Ph, the C₁₋₂₀ alkyl residues, preferably —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, and A represents an anion which is preferably selected from chloride, bromide, iodide or methosulfate.

Cationic silicone oils such as for example the commercially available Dow Corning 929 Emulsion (including a hydroxylamino-modified silicone, which is known as amodimethicone), DC 2-2078 (manufacturer: Dow Corning, INCI name: Aminopropyl Phenyl Trimethicone), DC 5-7113 (manufacturer: Dow Corning, INCI name: Silicone Quaternium 16), SM-2059 (manufacturer: General Electric), SLM-55067 (manufacturer: Wacker) and Abil®-Quat 3270 and 3272 (manufacturer: Th. Goldschmidt; diquaternary polydimethylsiloxanes, Quaternium-80) are suitable according to the invention.

Particularly preferred agents according to the invention are characterized in that they include at least one amino-functional silicone of formula (Si-3a)

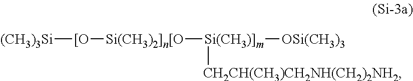

in which m and n are numbers whose sum (m+n) is between 1 and 2000, preferably between 50 and 150, wherein n preferably assumes values from 0 to 1999 and in particular from 49 to 149 and m preferably assumes values from 1 to 2000, in particular from 1 to 10.

Under the INCI declaration these silicones are known as trimethylsilylamodimethicones and they are available for example under the name Q2-7224 (manufacturer: Dow Corning; a stabilized trimethylsilylamodimethicone).

Also particularly preferred are agents according to the invention that include an amino-functional silicone of formula (Si-3b)

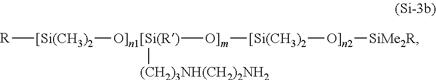

in which
R denotes —OH, an (optionally ethoxylated and/or propoxylated) (C₁ to C₂₀)alkoxy group or a —CH₃ group,
R′ denotes —OH, a (C₁ to C₂₀)alkoxy group or a —CH₃ group, and
m, n1 and n2 are numbers whose sum (m+n1+n2) is between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

Under the INCI declaration these silicones are known as amodimethicones or as functionalized amodimethicones, such as for example Bis(C13-15 Alkoxy) PG Amodimethicone (available for example as the commercial product DC 8500 from Dow Corning), Trideceth-9 PG-Amodimethicone (available for example as the commercial product Silcare Silicone SEA from Clariant).

Irrespective of which amino-functional silicones are used, cosmetic or dermatological preparations according to the invention are preferred which include an amino-functional silicone whose amine value is above 0.25 meq/g, preferably above 0.3 meq/g and in particular above 0.4 meq/g. The amine value denotes the milli-equivalents of amine per gram of the amino-functional silicone. It can be determined by titration and also specified in the unit mg KOH/g.

Hair treatment agents that are preferred according to the invention are characterized in that they include, relative to their weight, 0.01 to 10 wt. %, preferably 0.1 to 8 wt. %, particularly preferably 0.25 to 7.5 wt. % and in particular 0.5 to 5 wt. % of amino-functional silicone(s).

The compositions according to the invention can also include at least one polyammonium-polysiloxane compound as the silicone. The polyammonium-polysiloxane compounds can be purchased for example from GE Bayer Silicones under the trade name Baysilone®. The products with the names Baysilone TP 3911, SME 253 and SFE 839 are preferred here. The use of Baysilone TP 3911 as the active component of the compositions according to the invention is most particularly preferred.

The polyammonium-polysiloxane compounds are used in the compositions according to the invention preferably in an amount from 0.01 to 10 wt. %, preferably 0.01 to 7.5, particularly preferably 0.01 to 5.0 wt. %, most particularly preferably from 0.05 to 2.5 wt. %, relative in each case to the total composition.

The cyclic dimethicones referred to under INCI as cyclomethicones can also be used to advantage according to the invention. Hair treatment agents according to the invention are preferred here which include at least one silicone of the formula (Si-4)

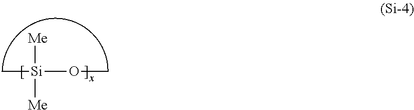

in which x denotes a number from 3 to 200, preferably from 3 to 10, more preferably from 3 to 7 and in particular 3, 4, 5 or 6.

The silicones described above have a backbone which is made up of —Si—O—Si units. These Si—O—Si units can of course also be interrupted by carbon chains. Corresponding molecules can be obtained by chain extension reactions and are preferably used in the form of silicone-in-water emulsions.

Agents that are likewise preferred according to the invention are characterized in that they include at least one silicone of formula (Si-5)

R₃Si—[O—SiR₂]ₓ—(CH₂)ₙ—[O—SiR₂]_{y}—O—SiR₃ (Si-5),

in which R denotes identical or different residues from the group —H, -phenyl, -benzyl, —CH₂—CH(CH₃)Ph, C₁-C₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y denote a number from 0 to 200, preferably from 0 to 10, more preferably from 0 to 7 and in particular 0, 1, 2, 3, 4, 5 or 6, and n denotes a number from 0 to 10, preferably from 1 to 8 and in particular 2, 3, 4, 5, 6.

As further silicones in addition to the dimethicones, dimethiconols, amodimethicones and/or cyclomethicones, water-soluble silicones can be included in the compositions according to the invention.

Corresponding hydrophilic silicones are selected for example from the compounds of formulae (Si-6) and/or (Si-7). Silicone-based water-soluble surfactants that are preferred in particular are selected from the group of dimethicone copolyols, which are preferably alkoxylated, in particular polyethoxylated or polypropoxylated.

According to the invention dimethicone copolyols are understood to be preferably polyoxyalkylene-modified dimethyl polysiloxanes of the general formulae (Si-6) or (Si-7):

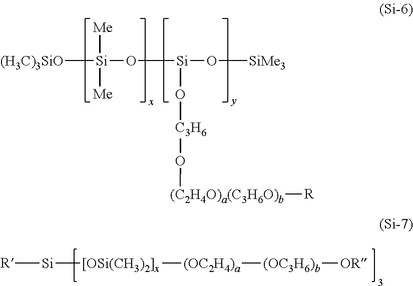

in which
the residue R denotes a hydrogen atom, an alkyl group having 1 to 12 C atoms, an alkoxy group having 1 to 12 C atoms or a hydroxyl group,
the residues R′ and R″ denote alkyl groups having 1 to 12 C atoms,
x denotes an integer from 1 to 100, preferably from 20 to 30,
y denotes an integer from 1 to 20, preferably from 2 to 10, and
a and b denote integers from 0 to 50, preferably from 10 to 30.

Particularly preferred dimethicone copolyols within the meaning of the invention are for example the products sold commercially under the trade name SILWET (Union Carbide Corporation) and DOW CORNING (Dow). Dimethicone copolyols that are particularly preferred according to the invention are Dow Corning 190 and Dow Corning 193 (Dow).

The dimethicone copolyols are preferably included in the compositions according to the invention in amounts from 0.01 to 10 wt. %, preferably 0.01 to 8 wt. %, particularly preferably 0.1 to 7.5 wt. % and in particular 0.1 to 5 wt. % of dimethicone copolyol, relative to the composition.

Finally, the silicone compounds are understood to include dimethiconols (Si8). Dimethiconols form a further group of silicones which are particularly preferred according to the invention. The dimethiconols according to the invention can be both linear and branched and also cyclic or cyclic and branched. Linear dimethiconols can be represented by the following structural formula (Si8-I):

(SiOHR¹₂)—O—(SiR²₂—O—)ₓ—(SiOHR¹₂) (Si8-I)

Branched dimethiconols can be represented by the structural formula (Si8-II):

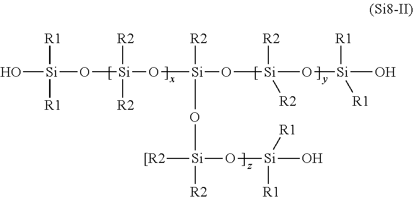

Residues R¹ and R² independently of one another denote hydrogen, a methyl residue, a C2 to C30 linear, saturated or unsaturated hydrocarbon residue, a phenyl residue and/or an aryl residue. Non-restricting examples of the residues represented by R¹ and R² include alkyl residues such as methyl, ethyl, propyl, isopropyl, butyl, isobutyl, pentyl, isopentyl, neopentyl, amyl, isoamyl, hexyl, isohexyl and similar; alkenyl residues, such as vinyl, halovinyl, alkyl vinyl, allyl, haloallyl, alkyl allyl; cycloalkyl residues, such as cyclobutyl, cyclopentyl, cyclohexyl and similar; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and similar, and sulfur-including residues, such as mercaptoethyl, mercaptopropyl, mercaptohexyl, mercaptophenyl and similar; R¹ and R² are preferably an alkyl residue including 1 to approximately 6 carbon atoms, and R¹ and R² are most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(O)OCH₂—, —(CH₂)₃ CC(O)OCH₂CH₂—, —C₆H₄C₆H₄—, —C₆H₄CH₂C₆H₄—; and —(CH₂)₃C(O)SCH₂CH₂—. Methyl, phenyl and C2 to C22 alkyl residues are preferred as R¹ and R². Of the C2 to C22 alkyl residues, lauryl, stearyl and behenyl residues are most particularly preferred. The numbers x, y and z are integers and each run independently of one another from 0 to 50,000. The molecular weights of the dimethiconols are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, particularly preferred viscosities are between 10,000 and 3,000,000 cPs. The most preferred range is between 50,000 and 2,000,000 cPs.

If the dimethiconols according to the invention are used as an emulsion, then according to the invention the droplet size of the emulsified particles is 0.01 to 10,000 μm, preferably 0.01 to 100 μm, most particularly preferably 0.01 to 20 μm and most preferably 0.01 to 10 μm. The particle size is determined by the light scattering method.

The following commercial products are cited as examples of such products: Dow Corning 1-1254 Fluid, Dow Corning 2-9023 Fluid, Dow Corning 2-9026 Fluid, X-21-5619 (Shin-Etsu Chemical Co.), Abil OSW 5 (Degussa Care Specialties), ACC DL-9430 Emulsion (Taylor Chemical Company), Dow Corning 1401 Fluid, Dow Corning 1403 Fluid, Dow Corning 1501 Fluid, Dow Corning 1784 HVF Emulsion, Dow Corning 9546 Silicone Elastomer Blend (all of the above Dow Corning Corporation), Silsoft 148, Silsoft E-50, Silsoft E-623 (all of the above Crompton Corporation), SM555, SM2725, SM2765, SM2785 (all of the above GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all of the above Wacker-Chemie GmbH).

The dimethiconols (Si8) are included in the compositions according to the invention preferably in amounts from 0.01 to 10 wt. %, preferably 0.01 to 8 wt. %, particularly preferably 0.1 to 7.5 wt % and in particular 0.1 to 5 wt. % of dimethiconol, relative to the composition.

If a mixture of at least two silicones is used, then this mixture is included in the compositions according to the invention in amounts from 0.01 to 10 wt. %, preferably 0.01 to 8 wt. %, particularly preferably 0.1 to 7.5 wt. % and in particular 0.1 to 5 wt. % of silicone mixture, relative to the composition.

In summary, hair treatment agents that are preferred according to the invention are characterized in that they include 0.15 to 15 wt. %, preferably 0.2 to 10 wt. %, more preferably 0.25 to 7.5 wt. %, still more preferably 0.5 to 5 wt. % and in particular 0.75 to 2.5 wt. % of at least one silicone from the group of dimethicones and/or cyclomethicones and/or amodimethicones and/or dimethiconols and/or trisiloxanes.

As a further ingredient the agents according to the invention can include one or more amino acids to particular advantage. Amino acids that can particularly preferably be used according to the invention derive from the group comprising glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cys), L-carnitine, L-citrulline, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the invention include one or more amino acids in relatively narrow quantity ranges. Hair treatment agents that are preferred according to the invention are characterized in that as a care substance they include—relative to their weight—0.01 to 5 wt. %, preferably 0.02 to 2.5 wt. %, particularly preferably 0.05 to 1.5 wt. %, more preferably 0.075 to 1 wt. % and in particular 0.1 to 0.25 wt. % of amino acid(s), preferably from the group comprising glycine and/or alanine and/or valine and/or lysine and/or leucine and/or threonine.

As a further constituent the agents according to the invention can include at least one carbohydrate from the group of monosaccharides, disaccharides and/or oligosaccharides. Hair treatment agents that are preferred according to the invention are characterized in that as a care substance they include—relative to their weight—0.01 to 5 wt. %, preferably 0.05 to 4.5 wt. %, particularly preferably 0.1 to 4 wt. %, more preferably 0.5 to 3.5 wt. % and in particular 0.75 to 2.5 wt. % of carbohydrate(s), selected from monosaccharides, disaccharides and/or oligosaccharides, wherein preferred carbohydrates are selected from
monosaccharides, in particular D-ribose and/or D-xylose and/or L-arabinose and/or D-glucose and/or D-mannose and/or D-galactose and/or D-fructose and/or sorbose and/or L-fucose and/or L-rhamnose,
disaccharides, in particular sucrose and/or maltose and/or lactose and/or trehalose and/or cellobiose and/or gentiobiose and/or isomaltose.

As has already been mentioned, preferred agents according to the invention include (an) amino acid(s).

Amino acids that can particularly preferably be used according to the invention derive from the group comprising glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cys), L-carnitine, L-citrulline, L-theanine, 3′,4′-dihydroxy-L-phenylalanine (L-dopa), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the invention include one or more amino acids in relatively narrow quantity ranges. Cosmetic agents that are preferred according to the invention are characterized in that they additionally include 0.05 to 5 wt. %, preferably 0.1 to 2.5 wt. %, particularly preferably 0.15 to 1 wt. % and in particular 0.2 to 0.5 wt % of amino acid(s), preferably (an) amino acid(s) from the group comprising glycine and/or alanine, and/or valine and/or lysine and/or leucine and/or threonine.

Surfactants are a particularly preferred group of ingredients.

Depending on the intended application, the content of surfactants from the individual groups can vary, with anionic surfactant(s) being particularly preferred in cleansing formulations (in particular in shampoos).

All anionic surface-active substances that are suitable for use on the human body are suitable as anionic surfactants and emulsifiers for the compositions according to the invention. These are characterized by a water-solubilizing anionic group such as for example a carboxylate, sulfate, sulfonate or phosphate group and a lipophilic alkyl group having approximately 8 to 30 C atoms. The molecule can additionally include glycol or polyglycol ether groups, ester, ether and amide groups and hydroxyl groups. Examples of suitable anionic surfactants and emulsifiers, each in the form of the sodium, potassium and ammonium salts as well as the mono-, di- and trialkanolammonium salts having 2 to 4 C atoms in the alkanol group, are
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
acyl glutamates of formula (I),

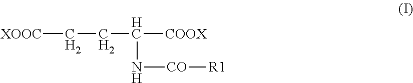

in which R¹CO denotes a linear or branched acyl residue having 6 to 22 carbon atoms and 0, 1, 2 or 3 double bonds and X denotes hydrogen, an alkali or alkaline-earth metal, ammonium, alkylammonium, alkanolammonium or glucammonium, for example acylglutamates derived from fatty acids having 6 to 22, preferably 12 to 18 carbon atoms, such as for example C_{12/14} or C_{12/18} coconut fatty acid, lauric acid, myristic acid, palmitic acid and/or stearic acid, in particular sodium-N-cocoyl and sodium N-stearoyl-L-glutamate,
esters of a hydroxyl-substituted di- or tricarboxylic acid of the general formula (II),

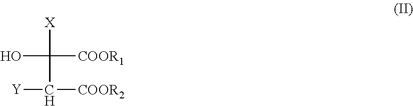

in which X═H or a —CH₂COOR group, Y═H or —OH on condition that Y═H if X═—CH₂COOR, R, R¹ and R² independently of one another denote a hydrogen atom, an alkali or alkaline-earth metal cation, an ammonium group, the cation of an organoammonium base or a residue Z, which derives from a polyhydroxylated organic compound selected from the group of etherified (C₆-C₁₈)alkyl polysaccharides having 1 to 6 monomeric saccharide units and/or the etherified aliphatic (C₆-C₁₆) hydroxyalkyl polyols having 2 to 16 hydroxyl residues, with the proviso that at least one of the groups R, R¹ or R² is a residue Z,
esters of sulfosuccinic acid or sulfosuccinates of the general formula (III),

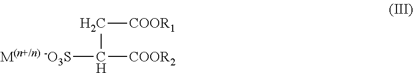

in which M^{(n+/n)} for n=1 is a hydrogen atom, an alkali metal cation, an ammonium group or the cation of an organoammonium base and for n=2 it is an alkaline-earth metal cation, and R¹ and R² independently of one another denote a hydrogen atom, an alkali or alkaline-earth metal cation, an ammonium group, the cation of an organoammonium base or a residue Z, which derives from a polyhydroxylated organic compound selected from the group of etherified (C₆-C₁₈)alkyl polysaccharides having 1 to 6 monomeric saccharide units and/or the etherified aliphatic, (C₆-C₁₆) hydroxyalkyl polyols having 2 to 16 hydroxyl residues, with the proviso that at least one of the groups R¹ or R² is a residue Z,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,
mixed surface-active hydroxyl sulfonates,
esters of tartaric acid and citric acid with alcohols which are addition products of approximately 2 to 15 molecules of ethylene oxide and/or propylene oxide with C₈₋₂₂ fatty alcohols,
alkyl and/or alkenyl ether phosphates,
sulfated fatty acid alkylene glycol esters,
monoglyceride sulfates and monoglyceride ether sulfates.

Preferred anionic surfactants are acyl glutamates, acyl isethionates, acyl sarcosinates and acyl taurates, each having a linear or branched acyl residue with 6 to 22 carbon atoms and 0, 1, 2 or 3 double bonds, which in particularly preferred embodiments is selected from an octanoyl, decanoyl, lauroyl, myristoyl, palmitoyl and stearoyl residue, esters of tartaric acid, citric acid or succinic acid or salts of these acids with alkylated glucose, in particular products with the INCI name Disodium Coco-Glucoside Citrate, Sodium Coco-Glucoside Tartrate and Disodium Coco-Glucoside Sulfosuccinate, alkyl polyglycol ether sulfates and ether carboxylic acids having 8 to 18 C atoms in the alkyl group and up to 12 ethoxy groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 ethoxy groups.

Irrespective of the type of anionic surfactants that are used, hair treatment agents according to the invention are preferred which include—relative to their weight—2.5 to 35 wt. %, preferably 5 to 30 wt. %, more preferably 7.5 to 27.5 wt. %, still more preferably 10 to 25 wt. % and in particular 12.5 to 22.5 wt. % of anionic surfactant(s).

Most particularly preferred agents according to the invention include fatty alcohol sulfates and/or fatty alcohol ether sulfates. Thus, hair treatment agents according to the invention which are characterized in that they include as an anionic surfactant—relative to their weight—0.1 to 20 wt. %, preferably 0.25 to 17.5 wt. % and in particular 2 to 15 wt. % of fatty alcohol sulfates of the formula

H₃C—(CH₂)ₙ—OSO₃⁻M⁺

in which n denotes values from 5 to 21, preferably from 7 to 19, particularly preferably from 9 to 17 and in particular from 11 to 13, and M denotes a cation from the group Na⁺, K⁺NH₄⁺, ½Mg²⁺, ½ Zn²⁺, preferably Na⁺, are preferred embodiments of the present invention.

Further preferred hair treatment agents according to the invention are characterized in that they include as an anionic surfactant—relative to their weight—0.1 to 20 wt. %, preferably 0.25 to 17.5 wt. % and in particular 2 to 15 wt. % of fatty alcohol ether sulfates of the formula

H₃C—(CH₂)ₙ—(OCH₂CH₂)ₖ—OSO₃⁻M⁺

in which n denotes values from 5 to 21, preferably from 7 to 19, particularly preferably from 9 to 17 and in particular from 11 to 13, and k denotes values of 1, 2, 3, 4, 5, 6, 7, 8, 9 or 10, preferably 1, 2 or 3 and in particular 2, and M denotes a cation from the group Na⁺, K⁺NH₄⁺, ½Mg²⁺, ½Zn²⁺, preferably Na⁺.

In addition to the anionic surfactants the agents according to the invention can include further surfactants.

The agents according to the invention include amphoteric surfactant(s) to particular advantage. Ampholytic surfactants and emulsifiers are understood to be surface-active compounds which in addition to a C₈-C₂₄ alkyl or acyl group include at least one free amino group and at least one —COOH or —SO₃H group and are capable of forming internal salts. Examples of suitable ampholytic surfactants are N-alkyl glycines, N-alkyl aminopropionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred ampholytic surfactants are N-cocoalkyl aminopropionate, cocoacyl aminoethyl aminopropionate and C₁₂-C₁₈ acyl sarcosine.

Particularly preferred hair treatment agents according to the invention are characterized in that they include amphoteric surfactant(s) from the groups of
N-alkyl glycines,
N-alkyl propionic acids,
N-alkyl aminobutyric acids,
N-alkyl iminodipropionic acids,
N-hydroxyethyl-N-alkylamidopropyl glycines,
N-alkyl taurines,
N-alkyl sarcosines,
2-alkyl aminopropionic acids each having approximately 8 to 24 C atoms in the alkyl group,
alkyl aminoacetic acids each having approximately 8 to 24 C atoms in the alkyl group,
N-cocoalkyl aminopropionate,
cocoacyl aminoethyl aminopropionate,
C₁₂-C₁₈ acyl sarcosine,
N-alkyl-N,N-dimethylammonium glycinates, for example cocoalkyl dimethylammonium glycinate,
N-acyl aminopropyl-N,N-dimethylammonium glycinates, for example cocoacyl aminopropyl dimethylammonium glycinate,
2-alkyl-3-carboxymethyl-3-hydroxyethyl imidazolines, each having 8 to 18 C atoms in the alkyl or acyl group,
cocoacyl aminoethyl hydroxyethyl carboxymethyl glycinate,
the compounds known under the INCI name Cocamidopropyl Betaine,
the compounds known under the INCI name Disodium Cocoamphodiacetate,
   preferred agents including the amphoteric surfactant(s) in amounts from 0.5 to 9 wt. %, preferably from 0.75 to 8 wt. % and in particular from 1 to 7.5 wt. %, relative in each case to the total agent.

Particularly preferred hair treatment agents include as amphoteric surfactants betaines of the formula (Bet-I)

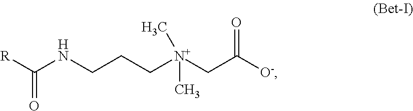

in which R denotes a straight-chain or branched, saturated or mono- or polyunsaturated alkyl or alkenyl residue having 8 to 24 carbon atoms.

Under INCI nomenclature these surfactants are known as Amidopropylbetaines, wherein the representatives derived from coconut fatty acids are preferred and are known as Cocoamidopropylbetaines. Surfactants of the formula (Bet-I) which are a mixture of the following representatives are particularly preferably used according to the invention:
H₃C—(CH₂)₇—C(O)—NH—(CH₂)₃N⁺(CH₃)₂CH₂COO⁻
H₃C—(CH₂)₉—C(O)—NH—(CH₂)₃N⁺(CH₃)₂ CH₂COO⁻
H₃C—(CH₂)₁₁—C(O)—NH—(CH₂)₃N⁺(CH₃)₂CH₂COO⁻
H₃C—(CH₂)₁₃—C(O)—NH—(CH₂)₃N⁺(CH₃)₂CH₂COO⁻
H₃C—(CH₂)₁₅—C(O)—NH—(CH₂)₃N⁺(CH₃)₂CH₂COO⁻
H₃C—(CH₂)₇—CH═CH—(CH₂)₇—C(O)—NH—(CH₂)₃N⁺(CH₃)₂CH₂COO⁻

Surfactants of the formula (Bet-I) are particularly preferably used within relatively narrow quantity ranges. Agents according to the invention are preferred here which—relative to their weight—include 0.25 to 8 wt. %, more preferably 0.5 to 7 wt. %, more preferably 0.75 to 6.5 wt. % and in particular 1 to 5.5 wt. % of surfactant(s) of the formula (Bet-I).

In addition to the amphoteric surfactant(s) of the formula (Bet-I) or in their place, the hair treatment agents according to the invention can include to particular advantage as amphoteric surfactants betaines of the formula (Bet-II)

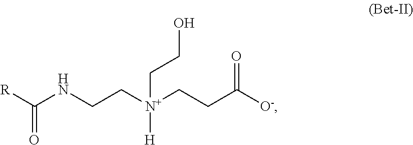

in which R denotes a straight-chain or branched, saturated or mono- or polyunsaturated alkyl or alkenyl residue having 8 to 24 carbon atoms.

Under INCI nomenclature these surfactants are known as Amphoacetates, wherein the representatives derived from coconut fatty acids are preferred and are known as Cocoamphoacetates.

For production reasons surfactants of this type always also include betaines of the formula (Bet-IIa)

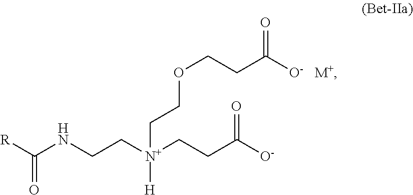

in which R denotes a straight-chain or branched, saturated or mono- or polyunsaturated alkyl or alkenyl residue having 8 to 24 carbon atoms and M denotes a cation.

Under INCI nomenclature these surfactants are known as Amphodiacetates, wherein the representatives derived from coconut fatty acids are preferred and are known as Cocoamphodiacetates.

Surfactants of the formula (Bet-II) which are a mixture of the following representatives are particularly preferably used according to the invention:
H₃C—(CH₂)₇—C(O)—NH—(CH₂)₂NH⁺(CH₂CH₂OH)CH₂CH₂COO⁻
H₃C—(CH₂)₉—C(O)—NH—(CH₂)₂NH⁺(CH₂CH₂OH)CH₂CH₂COO⁻
H₃C—(CH₂)₁₁—C(O)—NH—(CH₂)₂NH⁺(CH₂CH₂OH)CH₂CH₂COO⁻
H₃C—(CH₂)₁₃—C(O)—NH—(CH₂)₂NH⁺(CH₂CH₂OH)CH₂CH₂COO⁻
H₃C—(CH₂)₁₅—C(O)—NH—(CH₂)₂NH⁺(CH₂CH₂OH)CH₂CH₂COO⁻
H₃C—(CH₂)₇—CH═CH—(CH₂)₇—C(O)—NH—(CH₂)₂NH⁺(CH₂CH₂OH)CH₂CH₂COO⁻

Surfactants of the formula (Bet-II) are particularly preferably used within relatively narrow quantity ranges. Agents according to the invention are preferred here which—relative to their weight—include 0.25 to 8 wt. %, more preferably 0.5 to 7 wt. %, more preferably 0.75 to 6.5 wt. % and in particular 1 to 5.5 wt. % of surfactant(s) of the formula (Bet-II).

Particularly preferred hair treatment agents according to the invention are characterized in that they include 1 to 30 wt. %, preferably 1.5 to 25 wt. %, more preferably 2 to 20 wt. %, still more preferably 2.5 to 15 wt. % and in particular 3 to 10 wt. % of amphoteric surfactant(s).

In addition to the amphoteric surfactants or in their place, the agents according to the invention can also include non-ionic surfactants.

Particularly preferred non-ionic surfactants are alkyl polyglycosides. Alkyl polyglycosides (APGs) are non-ionic surfactants produced entirely from sustainable raw materials (sugar structural units, predominantly glucose, e.g. from corn starch, and fatty alcohol, e.g. from coconut oil). Alkyl polyglycosides are obtainable by acid-catalyzed reaction (Fischer reaction) of sugars, in particular glucose (or starch), or of butyl glycosides with fatty alcohols.

This gives rise to complex mixtures of alkyl monoglucoside (alkyl α-D and β-D glucopyranoside and small proportions of alkyl glucofuranoside), alkyl diglucosides (isomaltosides, maltosides, etc.) and alkyl oligoglucosides (maltotriosides, maltotetraosides, etc.). The average degree of polymerization of commercial products whose alkyl residues are in the range from C8 to C16, is 1.2 to 1.5.

Alkyl polyglycosides corresponding to the general formula RO—(Z)ₓ in which R denotes alkyl, Z denotes sugar and x denotes the number of sugar units, are preferably used according to the invention.

Alkyl polyglycosides in which R consists
substantially of C₈ and C₁₀ alkyl groups,
substantially of C₁₂ and C₁₄ alkyl groups,
substantially of C₈ to C₁₆ alkyl groups or
substantially of C₁₂ to C₁₆ alkyl groups or
substantially of C₁₆ to C₁₈ alkyl groups
   are particularly preferred.

Any mono- or oligosaccharides can be used as the sugar structural unit Z. Sugars having 5 or 6 carbon atoms and the corresponding oligosaccharides are conventionally used. Such sugars are for example glucose, fructose, galactose, arabinose, ribose, xylose, lyxose, allose, altrose, mannose, gulose, idose, talose and sucrose. Preferred sugar structural units are glucose, fructose, galactose, arabinose and sucrose. Preferred hair treatment agents according to the invention are thus characterized in that they include—relative to their weight—0.1 to 20 wt. %, preferably 1 to 10 wt. % and in particular 2 to 8 wt. % of alkyl polyglycoside(s) of the formula

H₃C—(CH₂)ₙ—O—(Z)ₓ

in which n denotes values from 5 to 21, preferably from 7 to 19, particularly preferably from 9 to 17 and in particular from 11 to 13, and k denotes values from 1.1 to 1.8, preferably from 1.2 to 1.5, and Z denotes a sugar structural unit from the group comprising glucose, fructose, galactose, arabinose, ribose, xylose, lyxose, allose, altrose, mannose, glucose, idose, talose and sucrose.

Glucose is a particularly preferred sugar structural unit (Z), such that preferred hair treatment agents according to the invention are characterized in that they include—relative to their weight—0.1 to 15 wt. %, preferably 1 to 10 wt. % and in particular 2 to 8 wt. % of alkyl polyglucoside(s) of the formula

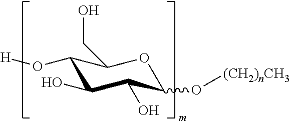

in which n denotes values from 5 to 21, preferably from 7 to 19, particularly preferably from 9 to 17 and in particular from 11 to 13, and m denotes values from 1.1 to 1.8, preferably from 1.2 to 1.5.

The alkyl polyglycosides for use according to the invention include on average 1.1 to 5 sugar units. Alkyl polyglycosides having x values from 1.1 to 2.0 are preferred. Alkyl glycosides in which x is 1.1 to 1.8 are most particularly preferred.

Most particularly preferred alkyl polyglucosides are those in which the alkyl residue is a lauryl residue. In the case of mixtures of substances from native sources, those sources having a high proportion of C12 fatty acids, in particular coconut fatty acids, are preferred. Particularly preferred hair treatment agents according to the invention are thus characterized in that they include—relative to their weight—0.1 to 15 wt. %, preferably 1 to 10 wt. % and in particular 2 to 8 wt. % of alkyl polyglucoside(s), in which n denotes the value 11, m denotes values from 1.1 to 1.8, preferably from 1.2 to 1.5.

For aesthetic reasons “clear” products are often preferred by consumers. Hair treatment agents that are preferred according to the invention are therefore characterized in that they are transparent or translucent.

Transparent or translucent within the context of the present invention is understood to be a composition having an NTU value of below 100. The NTU (nephelometric turbidity unit) value is a unit for turbidity measurements in liquids that is used in water treatment. It is the unit for a turbidity of a liquid measured with a calibrated nephelometer.

The agents according to the invention can moreover include a 2-pyrrolidinone-5-carboxylic acid and derivatives thereof (J). The sodium, potassium, calcium, magnesium or ammonium salts are preferred, in which the ammonium ion bears one to three C₁ to C₄ alkyl groups in addition to hydrogen. The sodium salt is most particularly preferred. The amounts used in the agents according to the invention are preferably 0.05 to 10 wt. %, relative to the total agent, particularly preferably 0.1 to 5, and in particular 0.1 to 3 wt. %.

It can additionally prove advantageous if penetration auxiliaries and/or swelling agents (M) are included in the agents according to the invention. They include for example urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as for example 1,2-propanediol, 1,2-pentanediol, 1,2-hexanediol, 1,2-dodecanediol, 1,3-propanediol, 1,6-hexanediol, 1,5-pentanediol, 1,4-butanediol.

Within the meaning of the invention short-chain carboxylic acids (N) can advantageously additionally support the active ingredient complex (A). Short-chain carboxylic acids and derivatives thereof are understood within the meaning of the invention to be carboxylic acids which can be saturated or unsaturated and/or straight-chain or branched or cyclic and/or aromatic and/or heterocyclic and which have a molecular weight of less than 750. Saturated or unsaturated straight-chain or branched carboxylic acids having a chain length of 1 to 16 C atoms in the chain can be preferred within the meaning of the invention, with those having a chain length of 1 to 12 C atoms in the chain being most particularly preferred.

Within the meaning of the invention the short-chain carboxylic acids can have one, two, three or more carboxyl groups. Carboxylic acids having more than one carboxyl group, in particular di- and tricarboxylic acids, are preferred within the meaning of the invention. The carboxyl groups can be present wholly or partially as the ester, acid anhydride, lactone, amide, imidic acid, lactam, lactim, dicarboximide, carbohydrazide, hydrazone, hydroxam, hydroxime, amidine, amidoxime, nitrile, phosphonic ester or phosphate ester. The carboxylic acids used according to the invention can of course be substituted along the carbon chain or the ring skeleton. The substituents of the carboxylic acids used according to the invention include for example C₁-C₈ alkyl, C₂-C₈ alkenyl, aryl, aralkyl and aralkenyl, hydroxymethyl, C₂-C₈ hydroxyalkyl, C₂-C₈ hydroxyalkenyl, aminomethyl, C₂-C₈ aminoalkyl, cyano, formyl, oxo, thioxo, hydroxyl, mercapto, amino, carboxyl or imino groups. Preferred substituents are C₁-C₈ alkyl, hydroxymethyl, hydroxyl, amino and carboxyl groups. Substituents in the α-position are particularly preferred. Most particularly preferred substituents are hydroxyl, alkoxy and amino groups, wherein the amino function can optionally be further substituted by alkyl, aryl, aralkyl and/or alkenyl residues. Preferred carboxylic acid derivatives are moreover likewise phosphonic and phosphate esters.

The present invention also provides a method for treating keratinic fibers, in particular human hair, wherein a hair treatment agent according to the invention is applied to keratinic fibers and either left there until the next hair wash (“leave-on” product) or rinsed off after a contact time from 30 to 900 seconds (“rinse-off” product).

The present invention therefore also provides a method for treating hair, wherein a hair treatment agent according to the invention is applied to the hair and rinsed off again after a contact time from 5 seconds to 15 minutes.

The present invention therefore also provides a method for treating hair, wherein a hair treatment agent according to the invention is applied to the hair and left there until the next hair wash.

All that has been stated in respect of the agents according to the invention applies with necessary alterations to preferred embodiments of the methods according to the invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent comprising, relative to its total weight,
a) at least one UV filter in a total amount from 0.01 to 20 wt. %
b) at least one compound of the general formula (I) in a total amount from 0.1 to 30 wt. % 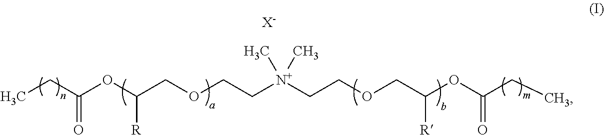
in which
n and m independently of one another denote integers between 5 and 40, with the proviso that n+m≧38;
a and b independently of one another denote integers between 1 and 10;
R and R′ are selected independently of one another from —H and —CH₃;
X⁻ is a physiologically acceptable anion.

2. The hair treatment agent according to claim 1, characterized in that, as a component of the at least one compound of the general formula (I), the agent includes 0.25 to 25 wt. % of at least one compound of the general formula (I).

3. The hair treatment agent according to claim 1, characterized in that, as a component of the at least one compound of the general formula (I), the agent includes 0.1 to 25 wt. % of at least one compound of the general formula (Ia) 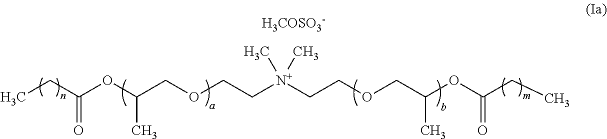
in which
n and m independently of one another denote integers between 5 and 40, with the proviso that n+m≧38;
a and b independently of one another denote 1, 2, 3, 4 or 5.

4. The hair treatment agent according to claim 1, characterized in that, as a component of the at least one compound of the general formula (I), the agent includes 0.5 to 20 wt. % of at least one compound of the general formula (Ib)

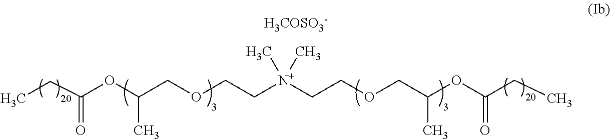

5. The hair treatment agent according to claim 1, characterized in that the at least one UV filter is selected from the group consisting of 2-hydroxy-4-methoxybenzophenone (benzophenone-3), 2-phenylbenzimidazole-5-sulfonic acid and the potassium, sodium and triethanolamine salts thereof (phenylbenzimidazole sulfonic acid), 3,3′-(1,4-phenylenedimethylene)bis(7,7-dimethyl-2-oxobicyclo-[2.2.1]hept-1-yl methanesulfonic acid) and salts thereof, 1-(4-tert-butylphenyl)-3-(4-methoxyphenyl)propane-1,3-dione (butyl methoxydibenzoylmethane), α-(2-oxoborn-3-ylidene)toluene-4-sulfonic acid and salts thereof, ethoxylated 4-aminobenzoic acid ethyl ester (PEG-25 PABA), 4-dimethylaminobenzoic acid-2-ethylhexyl ester (octyl dimethyl PABA), salicylic acid-2-ethylhexyl ester (octyl salicylate), 4-methoxycinnamic acid isopentyl ester (isoamyl p-methoxycinnamate), 4-methoxycinnamic acid-2-ethylhexy ester (octyl methoxycinnamate), 2-hydroxy-4-methoxybenzophenone-5-sulfonic acid and the sodium salt thereof (benzophenone-4), 2,2′,4,4′-tetrahydroxybenzophenone (benzophenone-2), 2,2′-dihydroxy-4,4′-dimethoxybenzophenone (benzophenone-6), cinnamic acid amidopropyl trimethylammonium chloride, and dodecyl dimethylaminobenzamidopropyl dimethylammonium tosylate and titanium dioxide.

6. The hair treatment agent according to claim 1, characterized in that the at least one UV filter includes a water-insoluble UV filter and a UV filter that includes a cationic group.

7. The hair treatment agent according to claim 1, characterized in that it includes titanium dioxide as the at least one UV filter.

8. The hair treatment agent according to claim 1, characterized in that it includes 0.15 to 15 wt. % of at least one ester of a carbonic acid selected from the group consisting of glycerol carbonate and dicaprylyl carbonate.

9. The hair treatment agent according to claim 1, characterized in that it includes 0.15 to 15 wt. % of at least one silicone from the group consisting of dimethicones, cyclomethicones, amodimethicones, dimethiconols, and trisiloxanes.

10. The hair treatment agent according to claim 1, characterized in that, as a component of the at least one compound of the general formula (I), the agent includes
a) 0.5 to 20 wt. % of at least one compound of the general formula (Ib) 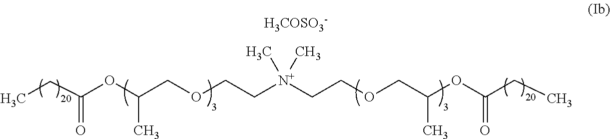
and
b) 0.1 to 10 wt. % of at least one compound of formula (IIa) [chemical structure]
